(19) United States
(12) Patent Application Publication
(10) Pub. No.: US 2019/0175381 A1
(43) Pub. Date: Jun. 13, 2019

(54) HEEL LIFT DEVICE

(71) Applicant: Melissa DeLany, Lake Worth, FL (US)

(72) Inventor: Melissa DeLany, Lake Worth, FL (US)

(21) Appl. No.: 16/212,299

(22) Filed: Dec. 6, 2018

Related U.S. Application Data
(60) Provisional application No. 62/595,860, filed on Dec. 7, 2017.

(51) Int. Cl. A61F 5/30

(52) U.S. Cl. CPC A61F 5/30

(57) ABSTRACT

A heel lift device includes an elongate resilient and cushioning body formed of an open cell polyurethane foam and having a top surface, a bottom surface, opposite side surfaces, and opposite end faces sloped at angled parallel planes to one another. In use, the bottom side is placed against the lower leg or ankle of the user and the elongate foam body is wrapped about the user's leg into a generally circular configuration so that at least a portion of the angled opposite end faces meet together. Next, the foam body is secured in place with one or more straps having releasable fasteners, such as hook and loop fasteners. The angled end faces allow for adjusted, uniform fitting to different size legs with no gaps. When fitted on the user's leg (or ankle), the device elevates the heel and eliminates pressure on the heel to prevent sores.

This non-provisional patent application is based on provisional patent application Ser. No. 62/595,860 filed Dec. 7, 2017.

### BACKGROUND OF THE INVENTION

### Field of the Invention

The present invention is directed to a heel lift device and, more particularly, to a heel lift device comprising an elongate foam body that wraps around a user's ankle or lower leg to form a generally donut configuration around the ankle or lower leg that helps to elevate the heel above an underlying surface and eliminate pressure on the heel, thereby preventing sores.

### Discussion of the Related Art

Many patients, particularly elderly patients, are confined to a bed for extended periods of time which may lead to a variety of sores and other complications. In particular, bed ridden patients are highly susceptible to heel pressure ulcers caused by the patients' heel rubbing against the mattress, bed structure and other objects.

In the past, various devices have been proposed to address the problem of heel sores and, particularly the need to elevate the heel above the underlying bed surface. Other devices have been proposed to elevate various parts of the body, including the arms and legs. Examples of related art devices include those disclosed in the U.S. Patent to Taylor et al., U.S. Pat. No. 7,188,382, directed to an ankle bolster, and Shields, U.S. Pat. No. 4,982,745, directed to a self elevating limb support. These devices disclosed in Taylor and Shields consist of a dense foam cylinder that is fitted to the lower leg (or arm) from just below the knee to the ankle. These devices allow the user to lie in a supine position while avoiding foot contact with the surface of the bed. However, a shortcoming of these devices is that they are designed to fit to a particular size leg. In particular, the device in Taylor has overlapping flanges on each end that are specifically designed to fit together and prevent one end from overrunning and creating a bulky or lumpy area. In the event the Taylor device were to be fitted to a larger person with thick legs, the device would leave a gap between the opposite ends. If fitted to a very thin leg, there would be some play or extra space as the flanges on the opposite ends meet together preventing the device from being tightened around the thin leg. Similarly the device in Shields does not allow for adjusted fitting to a person having very large legs or very thin legs. The device in Shields is designed to fit only a very specific sized leg. Therefore, the devices in Shields and Taylor would need to be provided in a range of sizes (e.g., small to XXL) to accommodate all users.

Accordingly, there remains a need for a foam device that wraps around a user's lower limb, and particularly the ankle or lower leg area, for purposes of elevating the heel to prevent contact and sores on the heel, and wherein the device is easily adjusted to accommodate a wide range of leg sizes without leaving gaps or space between the foam device and the user's leg.

### SUMMARY OF THE INVENTION

The present invention is directed to a heel lift device for elevating a person's heel above a surface, such as a bed surface. In particular, the heel lift device includes an elongate resilient and cushioning body formed of an open cell polyurethane foam and having a generally square or rectangular transverse cross-section, and further including a top surface, a bottom surface, opposite side surfaces, and opposite end faces that are sloped at an angle relative to the top and bottom surfaces. The opposite end faces are angled along parallel planes relative to one another with the angle between one of the end faces and the top surface being greater than 90 degrees and the angle of the other (opposite) end face and the top surface being less than 90 degrees. In use, the bottom side is placed against the lower leg or ankle of the user and the elongate foam body is wrapped about the user's leg into generally circular or donut configuration so that at least a portion of the angled opposite end faces meet together. Next, the foam body is secured in place with one or more straps having releasable fasteners, such as hook and loop fasteners. The angled end faces allow for adjusted, uniform fitting to different size legs with no gaps between the abutting end faces and no gaps between the bottom side of the device and the user's leg. When fitted on the user's leg (or ankle), the device elevates the heel and eliminates pressure on the heel to prevent sores.

### BRIEF DESCRIPTION OF THE DRAWINGS

For a fuller understanding of the nature of the present invention, reference should be made to the following detailed description taken in conjunction with the accompanying drawings in which:

FIG. 1 is a side elevational view showing the heel lift device of the present invention fitted to a user's lower leg or ankle and supporting the user's heel above a bed surface so as to prevent direct contact of the heel with the bed or other objects;

FIG. 2 is a side elevational view of the heel lift device of the present invention;

FIG. 3 is a top plan view of the heel lift device of the present invention showing an arrangement of straps with hook and loop fasteners for securing the heel lift device around the user's lower leg or ankle, as seen in FIG. 1; and

FIG. 4 is a side elevational view illustrating two of the heel lift devices of the present invention connected end to end in order to provide a longer overall length to accommodate exceptionally large legs or to fit over an immobilizer device or other device worn on the user's leg.

Like reference numerals refer to like parts throughout the several views of the drawings.

### DETAILED DESCRIPTION OF THE PREFERRED EMBODIMENT

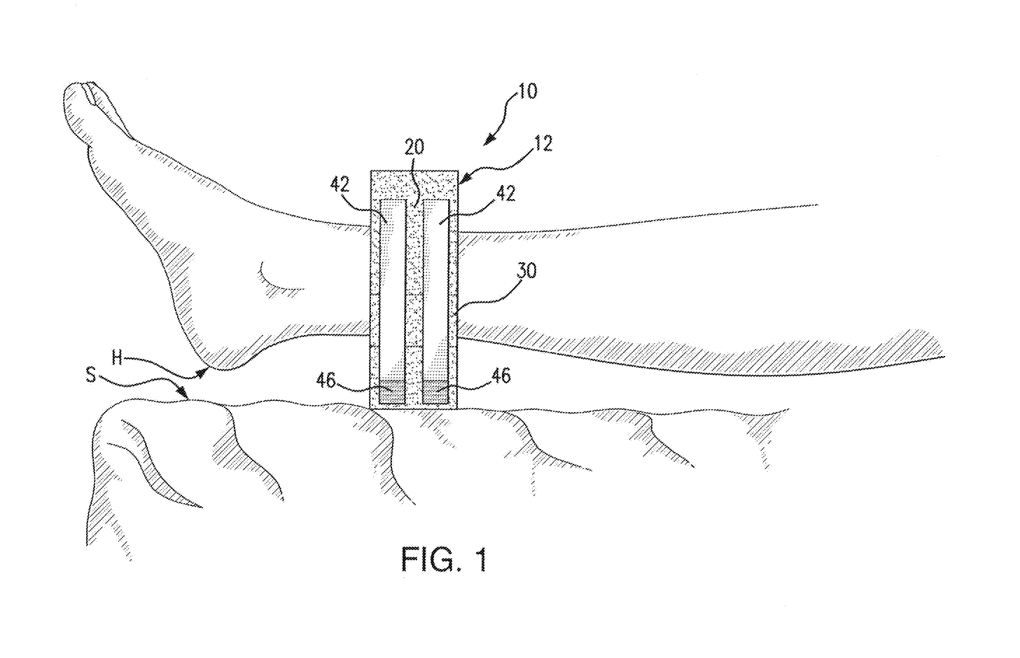

Referring to the several views of the drawings, and initially FIG. 1, the heel lift device of the present invention is shown and is generally indicated as 10. The heel lift device 10 is specifically designed to be fitted about a user's lower leg or ankle, as seen in FIG. 1, so that a user's heel H is elevated above a surface S, such as a bed surface, while the user is lying in a supine position. This helps to avoid contact of the user's heel H with the bed surface S or other objects, thereby eliminating pressure on the heel and preventing sores, particularly for bedridden patients.

The heel lift device 10 includes an elongate body 12 formed of a resilient and cushioning material. In one embodiment, the elongate body 12 is formed of an open cell polyurethane foam material that is latex free. In a preferred embodiment, the open cell polyurethane foam is breathable, and has a density of 2.8 lbs per cubic foot. The preferred embodiment of the device further provides for a polyurethane foam having 50 lb ILD (Indentation Load Deflection).

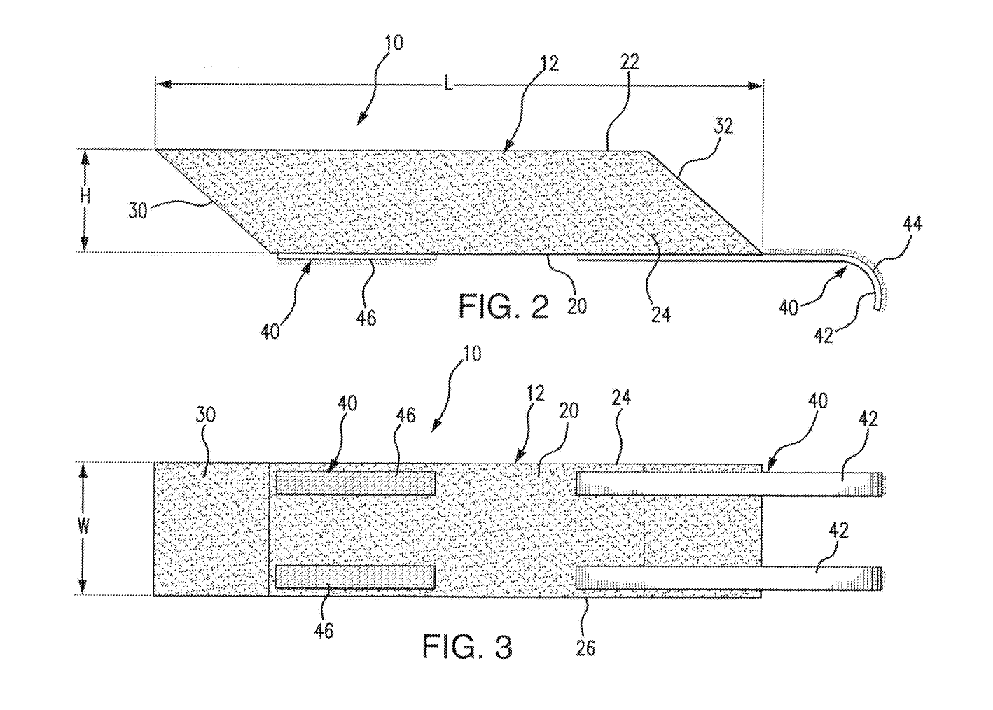

As seen in FIGS. 2 and 3, the elongate foam body 12 of the heel lift device 10 has a generally square or rectangular transverse cross-section with a flat top surface 20, a flat bottom surface 22 and opposite flat side surfaces 24 and 26. Further, the opposite end faces 30,32 of the elongate foam body are sloped at an angle relative to the top and bottom surfaces and are preferably angled along parallel planes relative to one another. Thus, as seen in FIG. 2, when viewed from a side elevation and with the elongate body in a relaxed state, the elongate foam body 12 has a shape that resembles a parallelogram, but not a rectangle.

The top surface 20 of the foam body 12 includes a releasable fastening system 40 for securing the foam body around the user's leg, as shown in FIG. 1. In a preferred embodiment, the releasable fastening system 40 includes a pair of elongate straps 42 that extend from one end of the foam body 12 on the top surface. The straps 42 include one component 44 of a releasable fastening system, such as a hook and loop fastening system. The opposite end of the top surface 20 of the foam body 12 is provided with cooperating releasable fastening components 46 that releasably attach to the components 44 on the straps 42 for adjustably securing the straps 42 thereto. In a preferred embodiment, the releasable fastening components 46 are one component of a hook and loop fastening system for releasable attachment to the hook and loop fastening component 44 on the respective cooperating strap 42.

In use, the bottom surface 22 of the elongate foam body 12 is placed against the user's lower leg or ankle and the foam body 12 is wrapped around the user's leg (or ankle) in a generally circular or donut configuration so that the foam body completely surrounds the user's leg. Once wrapped in this configuration, the foam body 12 is secured in place with the releasable fastening system 40. Specifically, the straps 42 are secured to the cooperating releasable fastening component 46 on the top surface 20 of the foam body, as seen in FIG. 1.

The angled end faces 30,32 are sloped relative to the top surface 20 and bottom surface 22. As seen in FIG. 2, the angle between end face 30 and top surface 20 is greater than 90 degrees, while the angle between end face 30 and bottom surface 22 is less than 90 degrees. Similarly, the angle between end face 32 and top surface 20 is less than 90 degrees while the angle between end face 32 and bottom surface 22 is greater than 90 degrees. The angled end faces 30,32 of the foam body 12 allow for adjusted, uniform fitting to different size legs with no gaps being left between the abutting opposite end faces 30,32 of the foam body 12 and no extra space between the foam body and the user's leg. When fitted on the user's leg (or ankle) the device 10 elevates the heel H, as shown in FIG. 1.

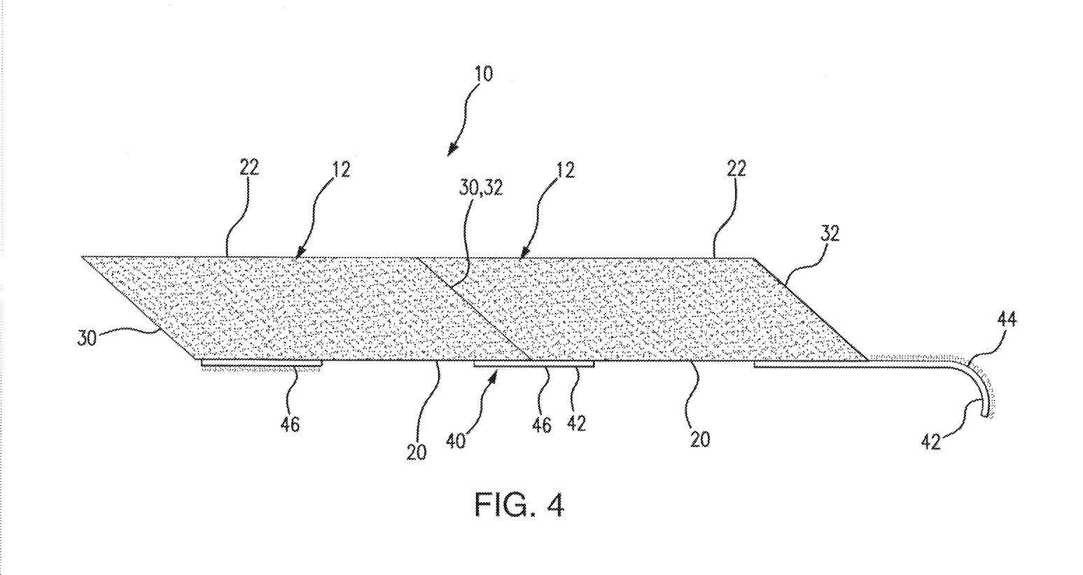

Referring to FIG. 4, two of the foam bodies 12 are shown attached end to end using the straps 42 on one foam body which attach to the releasable fastening components 46 on the other foam body. This allows for a greater overall length that can be completely wrapped around larger legs or an immobilizer device or other device worn on the user's leg.

While the present invention has been shown and described in accordance with a preferred and practical embodiment thereof, it is recognized that departures from the instant disclosure are fully contemplated within the spirit and scope of the present invention.

What is claimed is:

1. A heel lift device comprising:
an elongate main body formed of a resilient cushioning material and including a top surface, a bottom surface, opposite side surfaces and opposite end faces that are positioned at parallel planes relative to one another, and the opposite end faces being sloped relative to the top surface and the bottom surface with one of the end faces arranged at an angle of greater than 90 degrees to the top surface and less than 90 degrees to the bottom surface and the other end face arranged at an angle of less than 90 degrees to the top surface and greater than 90 degrees to the bottom surface;
the elongate main body being structured and disposed to be wrapped about a user's lower leg with the bottom surface held against the user's lower leg and the opposite end faces held in abutting engagement with one another with no gaps between the opposite end faces to define an operable position of the heel lift device on the user; and
the heel lift device being structured and disposed to rest on an underlying surface and support the user's entire foot, including the user's heel, elevated above the underlying surface so that the user's heel does not contact the underlying surface.

2. The heel lift device as recited in claim 1 wherein the main body is formed of an open cell polyurethane foam material.

3. The heel lift device as recited in claim 2 wherein the polyurethane foam material is latex free.

4. The heel lift device as recited in claim 2 wherein the polyurethane foam material has a density of 2.8 pounds per cubic foot.

5. The heel lift device as recited in claim 1 wherein the main body has a square transverse cross section and the top surface, the bottom surface and the opposite side surfaces are flat.

6. The heel lift device as recited in claim 1 wherein the main body has a rectangular transverse cross section and the top surface, the bottom surface and the opposite side surfaces are flat.

7. The heel lift device as recited in claim 1 wherein the at least one releasable securing member is at least one strap that is releasably secured to hold the heel lift device in the operable position.

8. The heel lift device as recited in claim 7 wherein the at least one releasable securing member includes at least one strap attached to and extending from the main body and terminating at a free distal end, and the free distal end being structured and disposed for releasable attachment to the main body to hold the heel lift device in the operable position.

9. The heel lift device as recited in claim 8 wherein the at least one strap includes one component of a hook and loop fastener thereon for releasable attachment to a cooperating component of a hook and loop fastener on the main body for allowing releasable attachment of the at least one strap to the main body to hold the heel lift device in the operable position.
